(19) United States
(12) Patent Application Publication
(10) Pub. No.: US 2025/0051519 A1
(43) Pub. Date: Feb. 13, 2025

(54) SOLUTION ELECTROSPUN FIBERS, COMPOSITIONS COMPRISING THE SAME AND A PROCESS OF MANUFACTURING THEREOF

(71) Applicant: UNIVERSITEIT GENT, Gent (BE)

(72) Inventors: Richard HOOGENBOOM, Terneuzen (NL); Chris VERVAET, Kachtem (BE); Jana BECELAERE, Lochristi (BE); Karen DE CLERCK, Afsnee (BE)

(73) Assignee: UNIVERSITEIT GENT, Gent (BE)

(21) Appl. No.: 18/722,254

(22) PCT Filed: Dec. 13, 2022
(86) PCT No.: PCT/EP2022/085640

§ 371 Date: Jun. 20, 2024

(30) Foreign Application Priority Data
Dec. 21, 2021 (EP) 21216446.1

(51) Int. Cl. C08G 73/02; D01D 5/00

(52) U.S. Cl. CPC C08G 73/0233; D01D 5/003

(57) ABSTRACT

The present invention relates to solution electrospun fibers comprising a ingredient in an amorphous form, compositions and their manufacturing process thereof. More specifically, the present invention relates to solution electrospun fibers and a compositions comprising at least one of said fibers, said fiber obtained by solution electrospinning a ingredient with a polymer.

### FIELD OF THE INVENTION

The present invention relates to the field of amorphous solid dispersions (ASDs). More precisely, the present invention relates to solution electrospun fibers comprising an ingredient in an amorphous form, compositions and their manufacturing process thereof. More specifically, the present invention relates to solution electrospun fibers and compositions comprising at least one of said fibers, said fiber obtained by solution electrospinning a ingredient with a polymer.

### BACKGROUND TO THE INVENTION

The development of oral dosage forms for poorly water-soluble active pharmaceutical ingredients (APIs) is a persistent challenge. A range of methods has been explored to address this issue, and amorphous solid dispersions (ASDs) have received increasing attention. ASDs are typically prepared by starting with a liquid precursor (a solution or melt) and applying energy for solidification. ASDs can be prepared in a variety of ways, namely by rapid cooling of a melt, precipitation of a drug-carrier solution or by direct solid conversion methods. These can be classified as heat-based methods, solvent-based methods and mechanochemical-based methods, respectively. Different preparation methods entail altered final product properties and an adequate rational selection of a manufacturing technology is indispensable. Heat-based methods apply the principle of rapid cooling of a melt as amorphization procedure and encompass spray congealing, melt granulation and hot-melt extrusion (HME). Such heat-based methods are only reliable for thermostable drugs but are of utmost importance regarding upscaling and continuous manufacturing purposes. Solvent-based methods use the intermediate step of a solution to convert the crystalline material into the amorphous state. Examples of these methods are film casting, bead coating, co-precipitation, freeze drying, electrospraying and spray drying. A huge benefit over the heat-based methods is that more thermolabile drugs can be processed. Mechanochemical activation (e.g. cryo-milling), which is a direct solid conversion technique, can also be used to prepare ASDs, but is still considered as nonconventional. Electrospinning has emerged in recent years as a potent option for the manufacturing of ASDs. This method uses electrical energy to induce changes from liquid to solid. Through the direct applications of electrical energy, electrospinning can generate nanofiber-based ASDs from drug-loaded solutions, melts and melt-solutions. The technique can also be combined with other approaches using the application of mechanical, thermal or other energy sources. Electrospinning has numerous advantages over other approaches to produce ASDs. These advantages include extremely rapid drying speeds, ease of implementation, compatibility with a wide range of ingredients (including those which are thermally labile), and the generation of products with large surface areas and high porosity. Furthermore, this technique exhibits the potential to create so-called ‘fifth-generation’ ASDs with nanostructured architectures, such as core/shell or Janus systems and their combinations. These advanced systems can improve dissolution behavior and provide programmable drug release profiles.

Electrospun fiber-based ASDs can maintain an incorporated ingredient in the amorphous physical form for prolonged periods of time because of their homogeneous drug distribution within the polymer matrix (typically they comprise solid solutions), and ability to inhibit molecular motion. These ASDs can be utilized to generate oral dosage forms for poorly water-soluble drugs, resulting in linear or multiple-phase release of one or more APIs. However, only a limited number of ASDs have reached the market due to low maximum drug loading contents in ASDs and their often low stability.

Vrbata et al., 2013, disclose solid dispersions of amorphous drugs in polymeric nanofibers. The prepared nanofibrous membranes disclosed in Vrbata et al., 2013, have been obtained by means of solution electrospinning and are provided to form nanofibrous membranes for sublingual administration of sumatriptan and naproxentherein for the treatment of migraine. Vrbata et al., 2013 disclose fibers having a drug load capacity of up to 40% of membrane mass. A drawback of fibers disclosed in Vrbata et al., 2013 is that the amount of active ingredient disclosed in said fibers is limited to 40% w/w.

Therefore, there is an industrial need for pharmaceutical compositions having increased drug loadings. The present invention provides for electrospun fiber-based ASDs and compositions comprising the same overcoming the drawbacks in the prior art.

### SUMMARY OF THE INVENTION

In a first aspect, the present invention relates to a process of manufacturing a solution electrospun fiber, wherein the process comprises the steps of:
a) providing a solution comprising an ingredient, a polymer and a solvent;

In other words, the present step provides for a solution of the ingredient and the polymer in said solvent.
b) electrospinning the solution of step a) thereby obtaining a solution electrospun fiber comprising an amorphous form of the ingredient.

In other words, the present step comprises electrospinning the solution obtained at step a) thereby obtaining an electrospun fiber. Wherein step a) comprises providing the polymer at a concentration which is lower than the concentration at which the polymer is solution electrospinnable in the absence of the ingredient.

Therefore, according to the present invention, step a) comprises providing the polymer at a concentration which is lower than the concentration at which the polymer is solution electrospinnable in the absence of the ingredient. In other words, at step a) the polymer is dissolved in the solvent and the concentration of the polymer in the solution thereby obtained is lower than the concentration required to obtain a electrospinnable solution without the ingredient. Expressed differently, in the absence of the ingredient the concentration of the polymer in the solvent is in itself insufficient to enable stable electrospinning of said solution resulting in fibers.

According to an embodiment of the present invention, at step a) the ingredient is provided in an amount of at least 50% w/w, preferably above 50% w/w of ingredient to polymer.

In a further embodiment of the present invention, at step a) the ingredient and the polymer are selected from those providing a mixture consisting of said ingredient and polymer yielding a solution electrospun fiber having a glass transition temperature T_{g} of at least about 25° C. In other words, the polymer and the ingredient are selected from those which mixture of the two provides a solution electrospun fiber with a glass transition temperature T_{g} of at least about 25° C. Therefore, in accordance with the present embodiment, the ingredient and the polymer are preferably selected based on their characteristics of being able to provide a mixture having such glass transition temperature. It has been found that a glass transition temperature of the mixture of the polymer and ingredient in accordance with the present embodiment provides for a better stability of the electrospun fibers obtained and the compositions therefrom. Therefore, in accordance with the present embodiment, the ingredient and the polymer are adapted to provide a mixture yielding a solution electrospun fiber with a T_{g} of at least about 25° C., wherein the T_{g} of the mixture is measured after said mixture is electrospun in the presence of an ingredient of choice.

In a further embodiment, at step a) the solvent provided comprises an acid, such as any one of: formic acid, acetic acid.

In a further embodiment of the present invention, not only the ingredient, but also the polymer is in an amorphous form in the electrospun fibers. An advantage of the present embodiment is that having an amorphous polymer leads to a more homogeneous distribution of the drug and enhanced polymer-drug interactions to keep the drug in the amorphous state. By utilization of amorphous polymers the dissolution of the amorphous ingredient, typically as a solid dispersion is enhanced. Moreover, it reduces the tendency of the ingredient, including API's to crystallize, by avoiding the presence of nucleation sites. In a further embodiment of the present invention, the polymer is a water-soluble polymer.

In an embodiment of the present invention, the polymer is a poly(2-alkyl-2-oxazoline) (PAOx), preferably poly(2-ethyl-2-oxazoline) (PEtOx). An advantage of the present embodiment is that the present polymer provides for the best electrospinnability characteristics and the highest obtainable drug loadings within the formed electrospun fibers.

In a second aspect, the present invention relates to a solution electrospun fiber obtainable according to the method of the present invention and any embodiment thereof comprising:
an ingredient in an amorphous form;
a polymer;
   wherein the solution electrospun fiber is electrospun from a polymer solution comprising a polymer at a concentration which is lower than the concentration at which the polymer is solution electrospinnable to form fibers in the absence of the ingredient.

A further embodiment of the present invention is directed to a fiber wherein the ingredient is in an amount of at least 50% w/w, preferably above 50% w/w of ingredient to polymer, preferably in an amount of at least 55% w/w, at least 60% w/w, at least 65% w/w, at least 70% w/w.

According to a further embodiment of the present invention, the ingredient and the polymer are selected from those providing a mixture consisting of said ingredient and polymer yielding a solution electrospun fiber having a glass transition temperature T_{g} of at least about 25° C.

In an embodiment of the present invention, the polymer in said fiber is an amorphous polymer and/or is water-soluble.

In a further embodiment of the present invention, the polymer is a poly(2-alkyl-2-oxazoline) (PAOx), preferably poly(2-ethyl-2-oxazoline) (PEtOx).

According to a further aspect, the present invention pertains to a composition comprising a solution electrospun fiber according to the present invention.

According to a further aspect, the present invention pertains to a solution electrospun fiber obtainable by means of the method defined in any one of the embodiment of the present description, comprising:
an ingredient in an amorphous form;
a polymer;
   wherein the ingredient is in an amount of at least 50%, preferably above 50% w/w of ingredient to polymer, preferably in an amount of at least 55% w/w, at least 60% w/w, at least 65% w/w, at least 70% w/w.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1, also abbreviated as FIG. 1, illustrates SEM images of solution electrospun fibers comprising PEtOx at different polymer weight percentages in the electrospinning solution.

FIG. 2, also abbreviated as FIG. 2, illustrates MDSC data of solution electrospun fibers in accordance with the present invention comprising itraconazole and PEtOx at different loadings of itraconazole. FIG. 3, also abbreviated as FIG. 3, illustrates XRD data of solution electrospun fibers in accordance with the present invention comprising itraconazole and PEtOx at different loadings of itraconazole. FIG. 4 also abbreviated as FIG. 4, illustrates dissolution data of solution electrospun fibers in accordance with the present invention comprising itraconazole and PEtOx at different loadings of itraconazole. FIG. 5, also abbreviated as FIG. 5, illustrates MDSC data of solution electrospun fibers in accordance with the present invention comprising celecoxib and PEtOx at different loadings of celecoxib. FIG. 6, also abbreviated as FIG. 6, illustrates XRD data of solution electrospun fibers in accordance with the present invention comprising celecoxib and PEtOx at different loadings of celecoxib. FIG. 7, also abbreviated as FIG. 7, illustrates XRD data of solution electrospun fibers in accordance with the present invention comprising flubendazole and PEtOx at different loadings of flubendazole. FIG. 8, also abbreviated as FIG. 8, illustrates dissolution data of solution electrospun fibers in accordance with the present invention comprising flubendazole and PEtOx at different loadings of flubendazole. FIG. 9, also abbreviated as FIG. 9, illustrates SEM images of solution electrospun fibers in accordance with the present invention comprising flubendazole and PVP K30 at different loadings of flubendazole. FIG. 10, also abbreviated as FIG. 10, illustrates SEM images of solution electrospun fibers in accordance with the present invention comprising flubendazole and PVP K90 at different loadings of flubendazole. FIG. 11, also abbreviated as FIG. 11, illustrates XRD data of solution electrospun fibers in accordance with the present invention comprising flubendazole and PEtOx at different loadings of flubendazole. FIG. 12, also abbreviated as FIG. 12, illustrates XRD data of solution electrospun fibers in accordance with the present invention comprising flubendazole and PEtOx at different storage durations. FIG. 13, also abbreviated as FIG. 13, illustrates the average plasma concentration of solution electrospun fibers in accordance with the present invention comprising flubendazole and PEtOx. FIG. 14, also abbreviated as FIG. 14, illustrates the AUC of solution electrospun fibers in accordance with the present invention comprising flubendazole and PEtOx.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. When describing the compounds of the invention, the terms used are to be construed in accordance with the following definitions, unless a context dictates otherwise.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

In a first aspect, the present invention relates to a process of manufacturing a solution electrospun fiber, comprising the steps of:
a) providing a solution comprising an ingredient, a polymer and a solvent;
b) electrospinning the solution provided at step a) thereby obtaining a solution electrospun fiber comprising an amorphous form of the ingredient;
   wherein step a) comprises providing the polymer at a concentration which is lower than the concentration at which the polymer is solution electrospinnable in the absence of the ingredient.

Therefore, a first step, step a), in the manufacturing of a solution electrospun fiber according to the present invention comprises providing a solution comprising an ingredient, a polymer and a solvent. In order to provide said solution, the ingredient and the polymer, either one before the other or vice-versa, can be dissolved in a selected solvent or mixture of solvents. The solvent shall be selected based on the required solubility and chemical characteristics of both the ingredient and the polymer, so to provide their full dissolution in said solvent, e.g. at the temperature at which the solution is electrospun. Subsequently, the solution obtained at step a) is electrospun, see step b). The electrospinning technique provides for the ingredient in an amorphous form, thereby creating an amorphous solid dispersion. Different setups can be used to carry out the electrospinning of the solution. An example electrospinning setup consists of a spinneret, which is typically a hypodermic syringe needle, which is connected to a high-voltage 5 to 50 kV direct current power supply, a syringe pump, and a grounded or negatively charged collector. The voltage to be set depends on several factors, such as the type of polymer and ingredient to be electrospun and the solvent. Further, it has been seen that the high voltage used in the electrospinning process has an impact on the diameter of the fibers to be obtained. Previous examples and prior art were limited to fibers comprising limited drug loading e.g. not exceeding 50 wt % for some drugs. A possible cause of this is the fact that higher drug loadings lead to higher viscosity solutions that cannot be electrospun. The present invention solves this problem, in particular, the inventors have surprisingly discovered that higher drug loading than the ones provided in literature can be obtained by lowering the polymer concentration below a critical threshold concentration, whereas this critical threshold concentration is the minimum required concentration of polymer in the polymer solution for which the polymer solution is electrospinnable into fibers without the ingredient being present. For example, in case a polymer solution not comprising the ingredient is electrospinnable into fibers at a concentration of the polymer within the solution from 25%, meaning that only from a concentration of 25% and higher it is possible to obtain uniform fibers, in accordance with the present invention, at step a) the polymer is provided at a concentration lower than 25%, because at a concentration lower than 25% the polymer is not solution electrospinnable into fibers without the ingredient.

In the context of the present invention, by means of the term “solution electrospinnable” or “electrospinnable”, reference is made to a polymer solution which is capable of providing uniform fibers by means of electrospinning said solution. The obtained fibers typically have a diameter in range from 50 nm to 5000 nm. In the context of the present invention, by means of the term “uniform fibers”, reference is made to fibers comprising less than 3% of beads, preferably less than 1% of beads and, a standard deviation of the fiber diameter which is less than 50%, less than 40%, less than 30%, less than 20%, wherein the percentage of beads is determined as percentage of beads area respect to the total material area, measured on SEM images. For example, non-electrospinnable solutions can be either solutions having a too high viscosity, which when trying to be electrospun are unable to provide for a time-stable jet, resulting in either no jet formation, or splashes of solution onto the collector, or can be solutions having a too low viscosity, which when trying to be electrospun provide a membrane containing large amounts of beads among the fibers resulting in a non-uniform membrane, or for even lower viscosities, solely beads are seen without the presence of fibers. The viscosity of solution is dependent on the polymer concentration.

In accordance with the present invention, step a) comprises providing the polymer at a concentration which is lower than the concentration at which the polymer is solution electrospinnable into fibers in the absence of the ingredient. In other words, at step a) the polymer is dissolved in the solvent and the concentration of the polymer in the solution thereby obtained is lower than the concentration required to obtain an electrospinnable solution without the ingredient.

The minimum concentration at which the solution is electrospinnable would depend on several parameters and most importantly on the kind of molecular interactions between the polymer, the solvent and the ingredient in solution.

The inventors have surprisingly found that electrospinning a solution comprising the polymer and the ingredient wherein the polymer is diluted compared to the solution wherein the polymer concentration is just enough for the electrospinning to occur, is beneficial in providing a higher concentration of ingredient in the obtained fiber compared to the prior art.

The solution electrospun fibers obtained can then be used in the manufacturing of compositions, e.g. pharmaceutical compositions. For example, the obtained fiber can subsequently be compacted with the addition of excipients, or it can simply be collected after electrospinning and/or encapsulated so to provide a composition to be administered.

In accordance with an embodiment of the present invention, at step a) the ingredient is provided in an amount of at least 50% w/w, preferably above 50% w/w of ingredient to polymer.

A first advantage of fibers provided by the present invention, their manufacturing method, and compositions thereof is previously unattainable drug loadings, based on literature reports as well as in house reference experiments using other more commonly applied formulation methods, such as solvent casting, HME and spray-drying. The inventors have surprisingly found that the use of solvent electrospinning enabled to more efficiently trap the drug in the amorphous form in the polymer matrix at high drug loadings.

In the context of the present invention, by means of the term “electrospinnable” or “solution electrospinnable”, reference is made to the possibility of obtaining at least one electrospun fiber from a solution, in other words, wherein the solution is capable of spinning.

In a further embodiment of the present invention the ingredient is provided in an amount of at least 55% w/w, at least 60% w/w, at least 65% w/w, at least 70% w/w.

In accordance with the present invention, the amount of the ingredient C_{ING}, (given as % w/w) also referred to as ingredient loading, or in case of the use of an active ingredient, is referred to as drug loading or drug content in case of an active pharmaceutical ingredient (API), is calculated according to the formula:

$${C}_{ING} = \frac{{m}_{ING}}{{m}_{total}} \times 100$$

wherein m_{ING} is the weight of the ingredient, mₜₒₜₐₗ is the weight of the components solubilized in chosen solvent to be electrospun, for example, in case the solution electrospun comprises only the ingredient and the polymer, mₜₒₜₐₗ would be equal to m_{ING}+m_{polymer}, wherein m_{polymer} is the weight of the polymer solubilized in the chosen solvent, in case e.g. the solution would comprise an additive, mₜₒₜₐₗ would be equal to m_{ING}+M_{polymer}+M_{additive} and so on.

If the drug loading is too high e.g. above its solubility limit in the polymer then crystallization may occur, meaning that for at least a portion of the drug there is an energy barrier to dissolution. The presence of some crystalline drug in the fibers led to their performing similarly to cast films in terms of drug release. Nevertheless, according to the method of the present invention, this can be avoided and compositions having both high drug loadings and amorphous character of the obtained fiber, allowing for both high drug loading and bioavailability.

In the context of the present invention, by means of the term “ingredient”, reference is made to a substance or mixture of substances intended to be used in the manufacture of a solution electrospun fiber. Ingredients which can be used in the context of the present invention can provide for fibers which can be used in various applications, such as food, cosmetic, personal care and medicaments. The ingredient present in the obtained solution electrospun fibers is therefore not limited to an active ingredient.

In the context of the present invention, by means of the term “active ingredient”, also referred to “active pharmaceutical ingredient” or “API”, reference is made to any substance or mixture of substances intended to furnish pharmacological activity or other direct effect in the diagnosis, cure, mitigation, treatment, or prevention of disease or to affect the structure and function of the body. The active ingredients used in the fibers of the present invention are any suitable active ingredients that are selected for treatment of the medical condition for which they are delivered, provided that they are either substantially insoluble in the polymers and solvents used in the fiber, or the amount of the drug exceeds the solubility limit of the drug in these materials.

General categories of active ingredients useful in the present invention include, but are not limited to: opioids; ACE inhibitors; adenohypophoseal hormones; adrenergic neuron blocking agents; adrenocortical steroids; inhibitors of the biosynthesis of adrenocortical steroids; alpha-adrenergic agonists; alpha-adrenergic antagonists; selective alpha-two-adrenergic agonists; androgens; anti-addictive agents; antiandrogens; antiinfectives, such as antibiotics, antimicrobals, and antiviral agents; analgesics and analgesic combinations; anorexics; antihelminthics; antiarthritics; antiasthmatic agents; anticonvulsants; antidepressants; antidiabetic agents; antidiarrheals; antiemetic and prokinetic agents; antiepileptic agents; antiestrogens; antifungal agents; antihistamines; antiinflammatory agents; antimigraine preparations; antimuscarinic agents; antinauseants; antineoplastics; antiparasitic agents; antiparkinsonism drugs; antiplatelet agents; antiprogestins; antipruritics; antipsychotics; antipyretics; antispasmodics; anticholinergics; antithyroid agents; antitussives; azaspirodecanediones; sympathomimetics; xanthine derivatives; cardiovascular preparations, including potassium and calcium channel blockers, alpha blockers, beta blockers, and antiarrhythmics; antihypertensives; diuretics and antidiuretics; vasodilators, including general coronary, peripheral, and cerebral; central nervous system stimulants; vasoconstrictors; hormones, such as estradiol and other steroids, including corticosteroids; hypnotics; immunosuppressives; muscle relaxants; parasympatholytics; psychostimulants; sedatives; tranquilizers; nicotine and acid addition salts thereof; benzodiazepines; barbiturates; benzothiadiazides; beta-adrenergic agonists; beta-adrenergic antagonists; selective beta-one-adrenergic antagonists; selective beta-two-adrenergic antagonists; bile salts; agents affecting volume and composition of body fluids; butyrophenones; agents affecting calcification; catecholamines; cholinergic agonists; cholinesterase reactivators; dermatological agents; diphenylbutylpiperidines; ergot alkaloids; ganglionic blocking agents; hydantoins; agents for control of gastric acidity and treatment of peptic ulcers; hematopoietic agents; histamines; 5-hydroxytryptamine antagonists; drugs for the treatment of hyperlipiproteinemia; laxatives; methylxanthines; monoamine oxidase inhibitors; neuromuscular blocking agents; organic nitrates; pancreatic enzymes; phenothiazines; prostaglandins; retinoids; agents for spasticity and acute muscle spasms; succinimides; thioxanthines; thrombolytic agents; thyroid agents; inhibitors of tubular transport of organic compounds; drugs affecting uterine motility; anti-vasculogenesis and angiogenesis; vitamins; and the like; or a combination thereof.

Some embodiments of the invention comprise an active ingredient that may include, but is not limited to: a) a corticosteroid, e.g., cortisone, hydrocortisone, prednisolone, beclomethasone propionate, dexamethasone, betamethasone, flumethasone, triamcinolone, triamcinolone acetonide, fluocinolone, fluocinolone acetonide, fluocinolone acetate, clobetasol propionate, or the like, or a combination thereof; b) an analgesic anti-inflammatory agent, e.g., acetaminophen, mefenamic acid, flufenamic acid, indomethacin, diclofenac, diclofenac sodium, alclofenac, ibufenac, oxyphenbutazone, phenylbutazone, ibuprofen, flurbiprofen, ketoprofen, salicylic acid, methylsalicylate, acetylsalicylic acid, 1-menthol, camphor, slindac, tolmetin sodium, naproxen, fenbufen, or the like, or a combination thereof; c) a hypnotic sedative, e.g., phenobarbital, amobarbital, cyclobarbital, lorazepam, haloperidol, or the like, or a combination thereof; d) a tranquilizer, e.g., fulphenazine, thioridazine, diazepam, flurazepam, chlorpromazine, or the like, or a combination thereof; e) an antihypertensive, e.g., clonidine, clonidine hydrochloride, bopinidol, timolol, pindolol, propranolol, propranolol hydrochloride, bupranolol, indenolol, bucumolol, nifedipine, bunitrolol, or the like, or a combination thereof; f) a hypotensive diuretic, e.g., bendroflumethiazide, polythiazide, methylchlorthiazide, trichlormethiazide, cyclopenthiazide, benzyl hydrochlorothiazide, hydrochlorothiazide, bumetanide, or the like, or a combination thereof; g) an antibiotic, e.g., penicillin, tetracycline, oxytetracycline, metacycline, doxycycline, minocycline, fradiomycin sulfate, erythromycin, chloramphenicol, or the like, or a combination thereof; h) an anesthetic, e.g., lydocaine, benzocaine, ethylaminobenzoate, or the like, or a combination thereof; i) another analgesic, e.g., acetylsalicylic acid, choline magnesium trisalicylate, acetaminophen, ibuprofen, celecoxib, fenoprofen, diflusinal, naproxen and the like; j) an antipruritic agent, e.g., bisabolol, oil of chamomile, chamazulene, allantoin, D-panthenol, glycyrrhetenic acid, a corticosteroid, an antihistamines and the like; k) an antimicrobial agent, e.g., methyl hydroxybenzoate, propyl hydroxybenzoate, chlorocresol, benzalkonium chlorides, nitrofurazone, nystatin, sulfacetamide, clotriamazole, or the like, or a combination thereof; l) an antifungal agent, e.g., itraconazole, pentamycin, amphotericin B, pyrrol nitrin, clotrimazole, or the like, or a combination thereof; m) a vitamin, e.g., vitamin A, ergocalciferol, cholecalciferol, octotriamine, riboflavin butyric acid ester, or the like, or a combination thereof; n) an antiepileptic, e.g., nitrazepam, meprobamate, clonazepam, or the like, or a combination thereof; o) an antihistamine, e.g., diphenhydramine hydrochloride, chlorpheniramine, diphenylimidazole, or the like, or a combination thereof; p) an antitussive, e.g., dextromethorphan, terbutaline, ephedrine, ephedrine hydrochloride, or the like, or a combination thereof; q) a sex hormone, e.g., progesterone, estradiol, estriol, estrone, or the like, or a combination thereof r) an antidepressant, e.g., doxepin; s) a vasodilator, e.g., nitroglycerin, isosorbide nitrate, nitroglycol, pentaerythritol tetranitrate, dipyridamole, or the like, or a combination thereof t) local anesthetics, e.g., procaine, benzocaine, chloroprocaine, cocaine, cyclomethycaine, dimethocaine/larocaine, propoxycaine, procaine/novocaine, proparacaine, tetracaine/amethocaine, lidocaine, articaine, bupivacaine, carticaine, cinchocaine/dibucaine, etidocaine, levobupivacaine, lidocaine/lignocaine, mepivacaine, piperocaine, prilocaine, ropivacaine, trimecaine, or the like; u) another drug, e.g., mebendazole, flubendazole, 5-fluorouracil, fenofibrate, dihydroergotamine, desmopressin, digoxin, methoclopramide, domperidone, scopolamine, scopolamine hydrochloride, or the like, or a combination thereof or the like; or a combination thereof.

Any opioid can be used in the embodiments of the present invention. Useful opioids include, but are not limited to, alfentanil, allylprodine, alphaprodine, anileridine, benzylmorphine, bezitramide, clonitazene, buprenorphine, butorphanol, codeine, desomorphine, dextromoramide, dezocine, diampromide, diamorphone, dihydrocodeine, dihydromorphine, dihydromorphone, dihydroisomorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl, heroin, hydrocodone, hydromorphone, hydromorphodone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, pantopon, papavereturn, paregoric, pentazocine, phenadoxone, phendimetrazine, phendimetrazone, phenomorphan, phenazocine, phenoperidine, piminodine, piritramide, propheptazine, promedol, properidine, propoxyphene, propylhexedrine, sufentanil, tilidine, tramadol, pharmaceutically acceptable salts thereof and mixtures of any two or more thereof.

According to an embodiment of the present invention, the ingredient is selected from the list comprising flubendazole, itraconazole, mebendazole, celecoxib and fenofibrate.

In accordance with the present invention, the ingredient is in an amorphous form. In the context of the present invention, by means of the term “amorphous form”, reference is made to a solid having substantial absence of long-range order. In other words, reference is made to a solid having a crystallinity lower than 5% at 25° C./25% relative humidity, preferably lower than 4%, preferably lower than 3%, preferably lower than 2%, preferably lower than 1%. Due to the absence of long-range order, amorphous materials are in an unstable (excited state) equilibrium, resulting in physical as well as chemical instability. The physical instability manifests itself in higher intrinsic aqueous solubility compared to the crystalline drug. The higher solubility of the amorphous drug leads to a higher rate of dissolution, and to better oral bioavailability.

In a further embodiment of the present invention, at step a) the ingredient and the polymer are selected from those providing a mixture consisting of said ingredient and polymer yielding a solution electrospun fiber having a glass transition temperature T_{g} of at least about 25° C. Therefore, in accordance with the present embodiment the ingredient and the polymer are preferably selected based on their being able to provide a fibre having such glass transition temperature. It has been found that a glass transition temperature of the fibers in accordance with the present embodiment provides for a better stability of the electrospun fibers obtained and the pharmaceutical composition therefrom.

In the context of the present invention, by means of the term “glass transition temperature”, reference is made to the temperature at which an amorphous solid transitions from a hard and relatively brittle “glassy” state into a viscous or rubbery state.

In a second aspect, the present invention relates to a a solution electrospun fiber comprising:
an ingredient in an amorphous form;
a polymer;
   wherein the solution electrospun fiber is electrospun from a polymer solution comprising a polymer at a concentration which is lower than the concentration at which the polymer is solution electrospinnable in the absence of the ingredient.

A further embodiment of the present invention is directed to a solution electrospun fiber wherein the ingredient is in an amount of at least 50% w/w of ingredient to polymer, preferably above 50% w/w of ingredient to polymer, such as at least 51% w/w, preferably in an amount of at least 55% w/w, at least 60% w/w, at least 65% w/w, at least 70% w/w.

In an embodiment of the present invention, the fiber comprises an ingredient and a polymer selected from those providing a mixture consisting of said ingredient and polymer yielding a solution electrospun fiber having a glass transition temperature T_{g} of at least about 25° C.

In an embodiment of the present invention, the polymer in said fiber is an amorphous polymer and/or is water-soluble.

In a further embodiment of the present invention, the polymer is a poly(2-alkyl-2-oxazoline) (PAOx), preferably poly(2-ethyl-2-oxazoline) (PEtOx).

In the context of the present invention, by means of the term “polymer”, reference is made to any suitable polymeric material. Several polymers can be used in accordance with the present invention. Examples of synthetic polymers that can be used in accordance with the present invention include biodegradable, bio-absorbable and no-biodegradable polymers, naturally derived or synthetic, such as, and not limited to, poly(2-alkyl-2-oxazoline)s, poly(2-ethyl-2-oxazoline) (PetOx), polyvinylpyrrolidone (PVP), polyvinylpyrrolidone-co-vinyl acetate (PVP-VA), crospovidone (PVPCL), polyvinyl alcohol (PVA), and polyethylene glycol (PEG); poly(8-caprolactone) (PCL), poly lactic-co-glycolic acid (PLGA), polyglycolic acid, poly(L-lactic acid), poly(DL-lactic acid); copolymers thereof such as poly(lactide-co-s-caprolactone), poly(glycolide-co-s-caprolactone), poly(lactide-co-glycolide), copolymers with polyethylene glycol (PEG); branched polyesters, such as poly(glycerol sebacate); poly(propylene fumarate); poly(ether esters) such as polydioxanone; poly(ortho esters); polyanhydrides such as poly(sebacic anhydride); polycarbonates such as poly(trimethylcarbonate) and related copolymers; polyhydroxyalkanoates such as 3-hydroxybutyrate, 3-hydroxy valerate and related copolymers that may or may not be biologically derived; polyphosphazenes; poly(amino acids) such as poly (L-lysine), poly (glutamic acid) and related copolymers; nylon4, 6; nylon 6; nylon 6,6; nylon 12; polyacrylic acid; polyacrylonitrile; poly(benzimidazole) (PBI); poly(etherimide) (PEI); poly(ethylenimine); poly(ethylene terephthalate); polystyrene; polysulfone; polyurethane; polyurethane urea; polyvinyl alcohol; poly(N-vinylcarbazole): polyvinyl chloride; poly (vinyl pyrrolidone); poly(vinylidene fluoride); poly(tetrafluoroethylene) (PTFE); polysiloxanes; and poly (methyl methacrylate). Examples of naturally occurring polymers or naturally derived polymers that can be used in accordance with the present invention include hydroxypropylmethyl cellulose (HPMC), hydroxypropylcellulose (HPC), hydroxypropylmethylcellulose phthalate (HPMCP) and hydroxypropylmethyl cellulose acetate succinate (HPMC-AS); and other types of naturally occurring polymers such as polypeptides such as collagen, elastin, albumin and gelatin; glycosaminoglycans such as hyaluronic acid, chondroitin sulfate, dermatan sulfate, keratan sulfate, heparan sulfate and heparin; chitosan and chitin; agarose; wheat gluten; polysaccharides such as starch, cellulose, pectin, dextran and dextran sulfate, trehalose, sucrose and inulin; and modified polysaccharides such as carboxymethylcellulose and cellulose acetate. Examples of other dissolvable or resorbable polymers include polyethylene glycol and poly(ethylene glycol-propylene glycol) copolymers that are known as pluronics and reverse pluronics. The function of the polymer is a stabilizing one, as the polymer helps with preventing the crystallization of the ingredient after the formulation has been electrospun and during dissolution in the body.

In a further embodiment of the present invention, the polymer is in an amorphous form in the fibers. An advantage of the present embodiment is that having an amorphous polymer leads to a more homogeneous distribution of the drug and enhanced polymer-drug interactions to keep the drug in the amorphous state. By utilization of amorphous polymers the dissolution of the amorphous solid dispersion is enhanced. Moreover, it reduces the tendency of the ingredient, including API's to crystallize, by avoiding the presence of nucleation sites.

In a preferred embodiment, the polymer is a poly(2-alkyl-2-oxazoline) (PAOx), preferably poly(2-ethyl-2-oxazoline) (PetOx). It has been found that poly(2-oxazolines) are particularly useful in stabilizing the ingredient and prevent of the ingredient, especially for drug loads above 50% w/w.

In accordance with a further embodiment of the present invention, the polymer is polyvinylpyrrolidone (PVP).

In a further embodiment of the present invention, the polymer is a water-soluble polymer. An advantage of the present embodiment is that the biocompatibility of the obtained fibers is improved and the drug solubilization is enhanced by dissolution of the polymer.

In the context of the present invention, by means of the term “polymer is water-soluble”, reference is made to a polymer having a solubility of at least about 1% w/w at 4° C.

In the context of the present invention, by means of the term “solvent”, reference is made to the solvent used in the formation of the polymer solution to be electrospun. Solvent choice is preferably based upon the solubility of the active agent and the polymer. Therefore, depending on both the ingredient and the polymer selected, suitable solvents for use herein include, but are not limited to acetic acid, acetone, acetonitrile, anisol, methanol, ethanol, propanol, ethyl acetate, propyl acetate, butyl acetate, butanol, N,N-dimethyl acetamide, N,N-dimethyl formamide, N-methyl-2-pyrrolidone, dimethyl sulfoxide, diethyl ether, diisopropyl ether, tetrahydrofuran, pentane, hexane, 2-methoxyethanol, formamide, formic acid, hexane, heptane, ethylene glycol, dioxane, 2-ethoxyethanol, trifluoroacetic acid, methyl isopropyl ketone, methyl ethyl ketone, dimethoxy propane, methylene chloride etc., or mixtures thereof.

In a further embodiment, at step a) the solvent provided comprises an acid, such as any one of: formic acid, acetic acid. In other words, in accordance with an embodiment of the present invention, the solvent provided at step a) is a solution comprising an acid, preferably an organic acid. The solvent can be provided by dissolution of organic acids e.g. formic acid and/or acetic acid or inorganic acids in another solvent, such as acetone. Solvents suitable to carry out the method according to the present invention include e.g. acetic acid, highly concentrated e.g. 100% acetic acid, acetone/acetic acid (8:2), formic acid e.g. highly concentrated formic acid, e.g. at least 98% formic acid. Although the polymer is water soluble, solvents are needed to ensure the dispersability and amorphous nature of the API. Solvents need to be selected on ensuring the ASD nature and electrospinnability.

In a preferred embodiment in accordance with the present invention, the solvent comprises formic acid. According to the present embodiment, the solvent comprises preferably at least 10% formic acid, at least 20% formic acid, at least 30% formic acid, at least 40% formic acid, preferably at least 50% formic acid, preferably at least 70% formic acid, preferably at least 90% formic acid, preferably at least 95% formic acid, preferably at least 98% formic acid. In accordance with the present embodiment, it has been found advantageous the use of a solution of formic acid has been found advantageous in providing the high API loadings.

In accordance with the present invention, the solvent should be selected among those capable of solubilizing a high amount of ingredient and the required amount of polymer needed for the solution electrospinning process. Preferably, as the desired end material is intended for internal use, a solvent with no/limited toxicity is required in case of residual solvent traces. By utilizing solvents, or solvent systems, that are classified as class Ill or IV by Impurities: Guideline for Residual Solvents (Q3C(R6)) of the International Council for Harmonization of Technical Requirements for Pharmaceuticals for Human Use the toxicological aspect is circumvented.

In the context of the present invention, by means of the term “solution electrospinning”, or “solution electrostatic spinning, reference is made to a process of producing fibers by applying a high voltage to a polymer solution to produce a polymer jet. As the jet travels in air, the jet is elongated under repulsive electrostatic force to produce fibers. It is believed that this electrospinning process, which involves extremely fast solvent evaporation, provides kinetic entrapment of the ingredient inside the polymeric matrix. The solution electrospinning technique does require the use of a solvent in order to obtain an electrospinnable solution. In accordance with the present invention, solvent should be preferably be removed from the finally achieved fiber. In case this is not achieved during the solution electrospinning, an additional drying step can be performed. Due to the necessity of a solution, there is a solubility limit. In order to obtain a homogeneous dispersion of the drug inside the polymeric fibers, a homogeneous solution is required. This can limit the maximal drug loading, however a switch to a more suited solvent or polymer can resolve the limited solubility e.g. another polymer or the same polymer but with a higher molecular weight.

A further aspect of the present invention relates to a composition comprising a solution electrospun fiber according to the present invention. Compositions according to the present invention comprise a fiber according to the present invention and a further component, such as an additive, such as, and not limited to, surfactants, absorption enhancers, flavouring agents, dyes, plasticizers, antioxidants. Etc. Examples of these further components are alginates, glycosaminoglycans (GAGs), water soluble gums including agar, arabic, carrageenans, cellulosics, chitin and chitosan based polymers, chondroitin sulfate, ethylene oxide containing polymers, poloxamers, ghatti, guars, hyaluronic acid, karaya, kadaya, locust bean, tragacanth, xantham, laminin, elastin, and other viscous media; Pluronicss (block copolymers of ethylene oxide and propylene oxide), lecithin, Aerosol OT™ (sodium dioctyl sulfosuccinate), sodium lauryl sulfate, Tween, such as Tween 20, 60 & 80, Span™, Arlacel™, Triton X-200, polyethylene glycols, glyceryl monostearate, Vitamin E-TPGS™ (d-alpha-tocopheryl polyethylene glycol 1000 succinate), sucrose fatty acid esters, such as sucrose stearate, sucrose oleate, sucrose palmitate, sucrose laurate, and sucrose acetate butyrate etc. and combinations thereof. Exemplary of plasticizers that may be employed in the fibers of this invention are triethyl citrate, triacetin, tributyl citrate, acetyl triethyl citrate, acetyl tributyl citrate, dibutyl phthalate, dibutyl sebacate, vinyl pyrrolidone and propylene glycol. Compositions according to the present invention can be used for various applications, including therapeutical applications, food applications, cosmetic, and personal care applications. In accordance with a further embodiment of the present invention, said composition is a pharmaceutical composition. In the context of the present invention, by means of the term “pharmaceutical composition”, reference is made to a medicinal product which includes an active ingredient, or active drug. Pharmaceutical compositions according to the present invention can be administered in accordance with different administration routes such as, and not limited to, oral, intravenous, intramuscular, or inhalation. The pharmaceutical compositions described comprise one or more pharmaceutically acceptable active agents or ingredients distributed within. Pharmaceutical compositions according to the present invention are preferably suitable to be used in a mammal, more preferably a human.

### EXAMPLES

In accordance with the present invention, solid dispersions containing five different active ingredients have been solution electrospun: fenofibrate, mebendazole, itraconazole, celecoxib and flubendazole. All of these active ingredients have been electrospun in high drug loadings equal to or higher than the loadings found in literature.

### Materials and Methods

### Materials

Defined PetOx with an Mn of 50 kDa was synthesized according to previous literature. [4] Polyvinylpyrrolidone (PVP), molecular weight 1000-1500 kDa (K90) and 44-54 kDa (K30) are available from BASF (Ludwigshafen, Germany). Drug substances such as Flubendazole (Flu), Mebendazole (MBZ), Itraconalzole (ITC), Celecoxib (CCX) and Fenofibrate (FFB) are commercially available from the manufacturer or various catalogs, such as UTAG B.V. (Almere, Netherlands). Solvents used such as Formic acid (FA) (>98%), Acetic acid (AA) (>99%), Acetone (Ac) (>99.5%), Anisole (>98%) and hydrochloric acid (HCl) (37%) are available from Sigma Aldrich (Overijse, Belgium) and used as such. All tests requiring an aqueous solution were carried out with distilled water of type III as considered in ISO Standard 3696.

### Methods

### Solvent Electrospinning

Electrospinning solutions were prepared by dissolving varying amounts of API in the optimal solvent system. Mass concentrations are expressed by weight percentages (wt %) defined for the polymer as the ratio of polymer mass and the sum of the polymer and solvent mass (Eq. (1)). For the amount of API in the system, the wt % is defined as the ratio of the mass of API to the sum of the API and polymer mass (Eq. (2)). All solution electrospinning experiments were carried out using a mononozzle, KD Scientific Syringe Pump Series 100, set-up with an 18 gauge Terumo mixing needle without bevel. A stable Taylor cone was achieved according to a tip-to-collector distance and a voltage (Glassman High Voltage Series EH-B) depending on the system. All electrospinning experiments were carried out under climatized conditions at 25° C. and 30% relative humidity in a Weisstechnik WEKK 10.50.1500 climate chamber. After electrospinning all samples were stored in a climatized lab at 23° C.±1° C. and a relative humidity of 25%+2%.

$$wt {\%}_{Polymer} = \frac{{m}_{Polymer}}{{m}_{Polymer} + {m}_{solvent}} \qquad ( 1 )$$

$$wt {\%}_{API} = \frac{{m}_{API}}{{m}_{API} + {m}_{Polymer}} \qquad ( 2 )$$

### Scanning Electron Microscopy

All produced membranes were analyzed on a Phenom XL Scanning Electron Microscope (SEM) at an accelerating voltage of 10 kV. Prior to analysis the samples were coated with gold using a sputter coater (LOT MSC1T). The membranes were investigated for irregularities such as beads. The fiber diameters were evaluated via the FiberMetric software. The average diameters and their standard deviations are based on 500 measurements per sample.

### Amorphous Nature and Time Stability

### Temperature Modulated Differential Scanning Calorimetry

Temperature Modulated Differential Scanning Calorimetry (MDSC) was used to measure glass transition temperatures (T_{g}). A TA Instruments Q2000 equipped with a refrigerated cooling system (RCS90) was applied using nitrogen as purge gas (50 mL·min⁻¹). Samples underwent a modulation of ±0.32° C./60 s at an average rate of 2° C./min. A heat cool heat procedure was applied for all samples at different temperature ranges depending on the material properties, e.g., degradation temperature. The instrument was calibrated using Tzero technology for standard Tzero aluminum pans using indium at the heating rate applied during the measurements. The T_{g} was evaluated with TA TRIOS software. Experiments were performed in triplicate.

### X-Ray Diffraction

ASDs, both nanofibrous and solvent casted, were measured on an ARL™ X'TRA Powder Diffractometer of Thermo Scientific. The monochromatic X-rays are produced by a copper X-ray tube; Cu K-shell energy levels are equivalent to λ=0.154056 nm. The diffraction patterns are recorded at an interval of 5 to 60° 2θ with a step size of 0.02° and a measuring time of 45 min. A Si (Li) solid state detector was used for data collection. The nanofibrous membranes were measured as such, while the solvent casted ones were grinded to a powder.

### In Vitro Dissolution Assay

The equipment used for these experiments is a double beam Perkin-Elmer Lambda 900 UV-Vis spectrophotometer. Solutions were measured in transmission mode using quartz cells. The spectra were recorded from 250 nm to 400 nm with a data interval of 1 nm.

For both pure API and the produced solid dispersions in vitro dissolution tests were performed in 0.1 M HCl. The medium was stirred at a constant speed of 100 rpm throughout the measurements and samples were taken at different time intervals, i.e. 3, 5, 10, 15, 30, 60, 90, 120, 180, 240 and 300 minutes. Samples were filtered through a 0.45 μm PTFE filter, diluted and consequently measured via UV-Vis spectroscopy. Experiments were performed in triplicate.

### In Vivo Dissolution Assay

Capsules of a 00 size were filled with either 50 wt % Flu-PEtOx nanofibrous material or with pure crystalline Flu. Each capsule contained 200 mg of Flu. These capsules were administered to beagles in 2 different sessions with at least 8 days in between both sessions. The study was approved by the ethical commission for animal testing at Ghent University. Before the dogs were fed the capsules, a fasting time of 12 hours was set. Using an intravenous catheter in the vena cephalica blood samples of 3 ml were collected at different time intervalls of 0.5, 1, 2, 4, 6, 8, 12, 16, 20 and 24 hours after administration. Plasma samples were stored at −20° C. until analyzed using an LC-MS method.

### Electrospinning of Pure Poly(2-Ethyl-2-Oxazoline)

PetOx is a polymer which is soluble in a variety of solvents such as water, formic acid, acetone and so on. In the following examples PetOx with a molecular weight of 50 kDa is used with a formic acid solvent system. PetOx is stably electrospinnable for wt % starting from 25 wt %, at wt % lower than this amount the viscosity in the solution is too low as a result of not enough chain entanglements. This results in non-uniform and beaded nanofibers as shown in FIG. 1a, once the wt %, and hence the amount of chain entanglements, is 25 wt % or more, uniform nanofibers are observed as seen in FIG. 1b. By solvent electrospinning solutions of either formic acid-PetOx and ethanol-PetOx with different wt %, i.e. 10, 15, 20, 25 and 30, and consequently evaluating the resulting samples via SEM analysis the minimal required polymer concentration of 25 wt % for the electrospinning of pure PetOx was established based on the absence of beads and uniformity of the nanofibers.

FIG. 1 illustrates SEM images comprising solution electrospun fibers in the absence of an active ingredient, which have been obtained by electrospinning pure PetOx. The provided data illustrates the necessity of a certain polymer concentration in the electrospinning solution. More specifically FIG. 1a illustrates the presence of beads in the membrane due to too low viscosity and polymer concentration at 15 wt % PetOx, which is below the minimal amount required for a stable and uniform solution electrospinning process. FIG. 1b illustrates a uniform solution electrospun nanofibrous membrane, electrospun from a 30 wt % PetOx solution. Based on the obtained data the solution electrospinning of pure PetOx is stable starting from a 25 wt % of PetOx in the solution.

### Example 1: Electrospinning of Poly(2-Ethyl-2-Oxazoline) with Mebendazole (MBZ)

Various amorphous solid dispersions were obtained by the solution electrospinning of PetOx and MBZ in a formic acid solvent system. Table 1 shows the ranges of high drug loadings that are obtained (X denotes an inhomogeneous solution). The process conditions were a tip to collector distance of 15 cm, a flow rate ranging between 0.1 and 0.15 ml/h and a voltage of 22.5-27.5 kV. Note that the high mebendazole loadings were only reached when a PetOx wt % was used which is lower compared to the minimal required polymer concentration for solution electrospinning pure PetOx.

TABLE 1

| Mebendazole | 30 wt % | 40 wt % | 50 wt % | 55 wt % | 60 wt % |
|---|---|---|---|---|---|
| 10 wt % PetOx | | | | | 186 ± 45 |
| | | | | | nm |
| 15 wt % PetOx | | 101 ± 32 | 213 ± 74 | 172 ± 39 | X |
| | | nm | nm | nm | |
| 25 wt % PetOx | 327 ± 101 | X | X | X | X |
| | nm | | | | |

Thermal analysis via mDSC shows no crystalline melting endotherm when heated from −20° C. till 120° C. Moreover, a single T_{g} is seen indicating a homogeneous, single phase material with glass transition temperatures ranging from 85° C. till 97° C., temperatures well above room temperature.

### Example 2: Electrospinning of poly(2-ethyl-2-oxazoline) with Itraconazole (ITC)

Nanofibrous amorphous solid dispersions containing ITC were obtained via solution electrospinning with PetOx as polymeric excipient. Table 2 shows that high drug loadings up to 70 wt % were achieved when an acetic acid solvent system was used (X denotes an inhomogeneous solution). Parameters used for the solvent electrospinning process are a tip to collector distance of 15 cm, a flow rate of 0.75 till 1 ml/h and a voltage between 15 and 20 kV. Note that the high itraconazole loadings were only reached when a PetOx wt % was used which is lower compared to the minimal required polymer concentration for solution electrospinning pure PetOx.

TABLE 2

| Itraconazole | 40 wt % | 50 wt % | 55 wt % | 60 wt % | 65 wt % | 70 wt % |
|---|---|---|---|---|---|---|
| 15 wt % | | | | | | 723 ± |
| PetOx | | | | | | 129 nm |
| 20 wt % | 489 ± | 576 ± | 565 ± | 996 ± | X | X |
| PetOx | 98 nm | 102 nm | 143 nm | 160 nm | | |

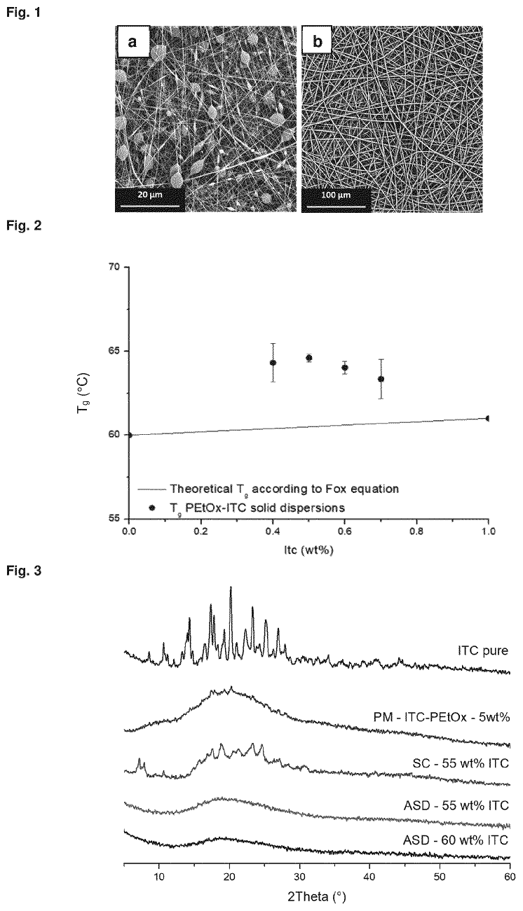

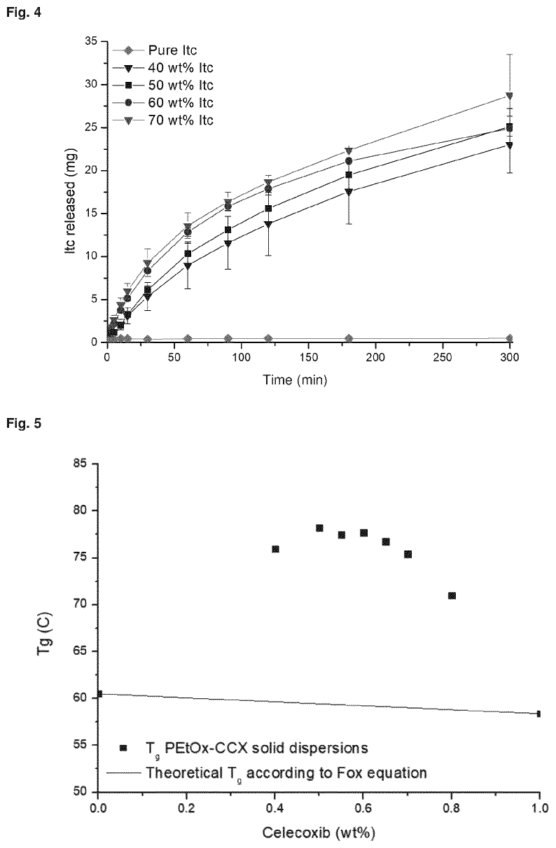

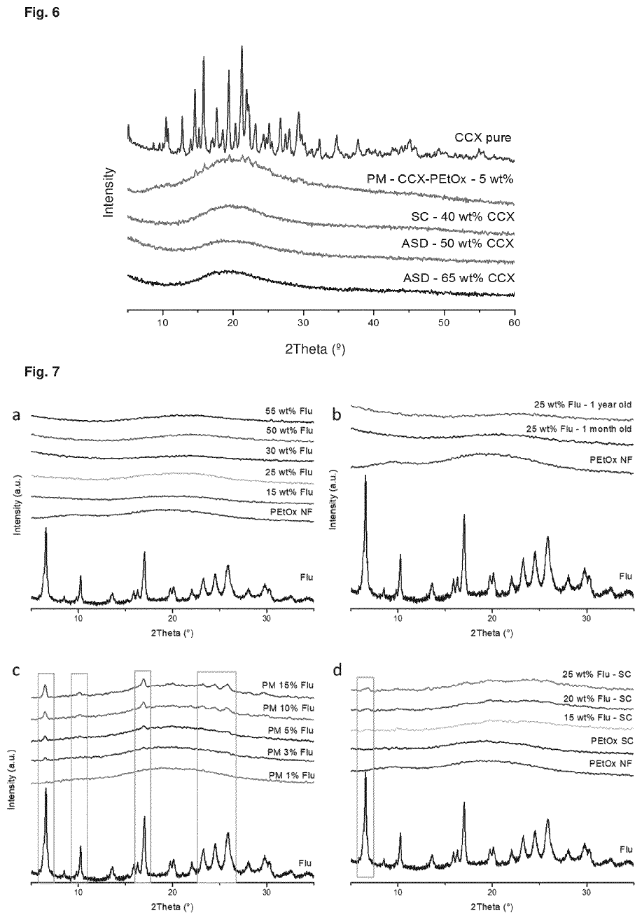

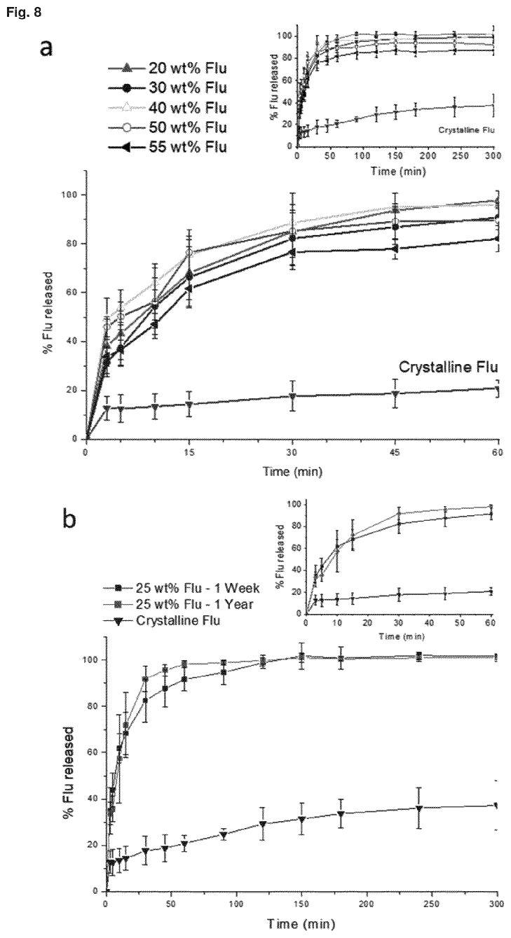

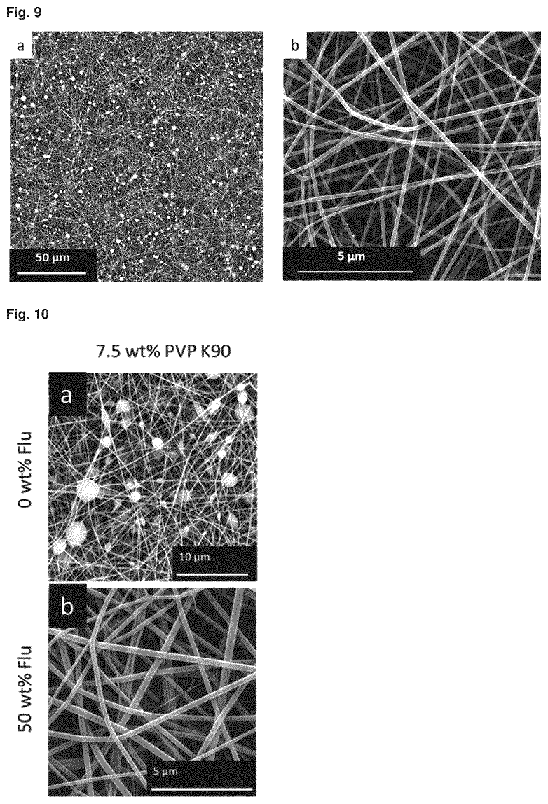

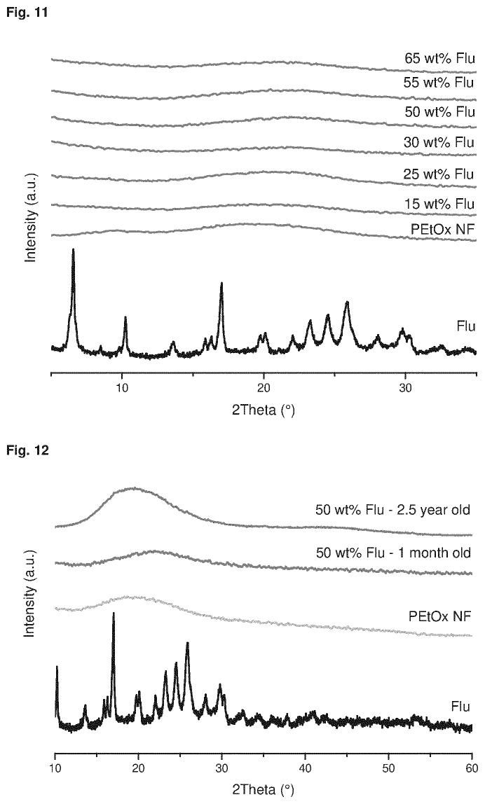

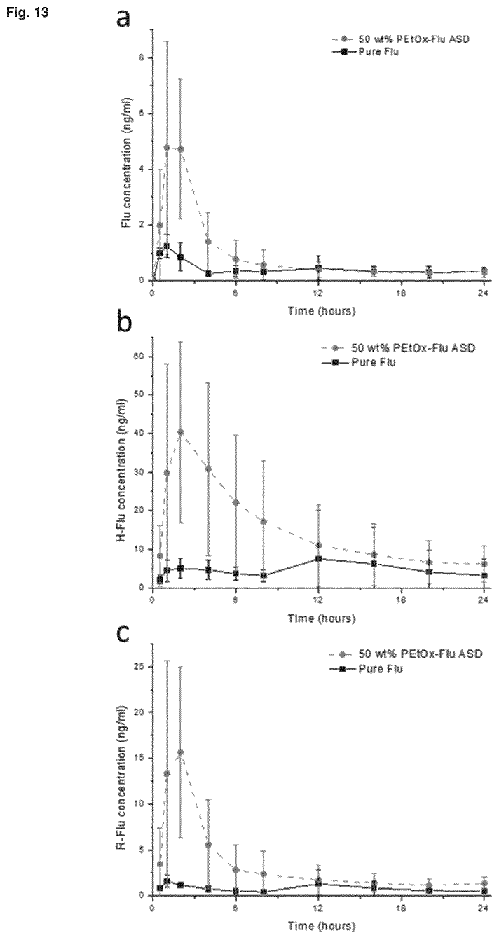

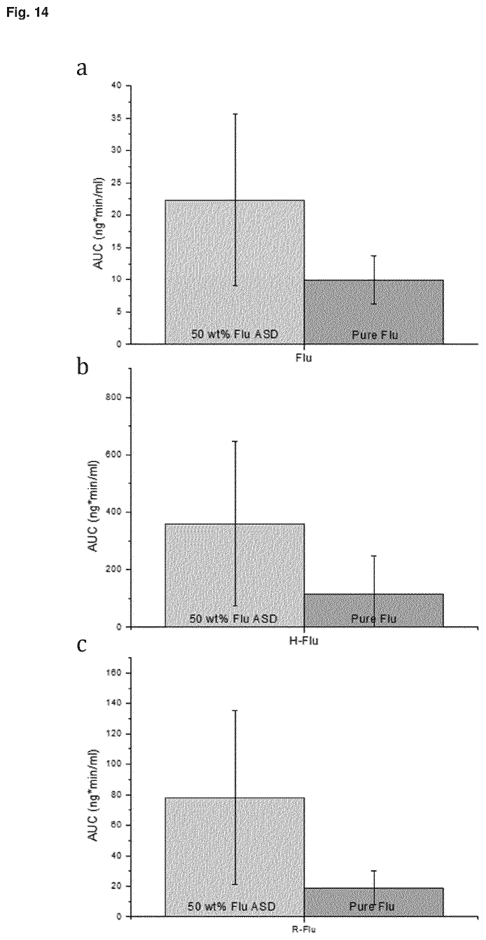

MDSC measurements clearly lack a crystalline endothermic peak in the temperature range applied, i.e., 0° C. till 200° C. A single T_{g} is observed at a temperature around 65° C. as shown in FIG. 2, which is well above room temperature. This indicates a homogeneous, single phase material, moreover, as the T_{g} is above the theoretical T_{g} as calculated by the mixing rule, Fox equation, this indicates a stability increasing interaction between PetOx and ITC.

In particular, FIG. 2 illustrates MDSC data of pharmaceutical compositions comprising solution electrospun fibers obtained in accordance with the present invention wherein itraconazole is the active ingredient and PetOx is the polymer. The provided MDSC data illustrates the T_{g} values for compositions at different loadings of itraconazole. More specifically, FIG. 2 illustrates the obtained T_{g} values (circles) compared to the theoretically predicted T_{g} values as predicted by the Fox equation for mixing (line). Based on the obtained data values above the prediction are obtained indicating a stability increasing interaction between PetOx and itraconazole.

Additionally, XRD analysis shown in FIG. 3 compares the diffraction pattern of pure crystalline ITC and a physical mixture of ITC and PetOx containing 5 wt % ITC to the patterns of both solvent electrospun solid dispersions and solvent casted (SC) solid dispersion. The SC samples are samples prepared from the same solution as the electrospinning solution, but instead of being solvent electrospun, they are left to a solvent evaporation process, which is distinctly slower compared to the rapid solvent evaporation of solvent electrospinning. The advantage of the electrospinning process is indicated by the lack of crystalline Bragg peaks, even the 60 wt % ITC sample indicates full amorphicity. Whereas the solvent casted membrane is already exhibiting partially crystalline ITC (>5 wt % as compared to the physical mixture).

Therefore, FIG. 3 illustrates XRD data of pharmaceutical compositions comprising solution electrospun fibers obtained in accordance with the present invention wherein itraconazole is the active ingredient and PetOx is the polymer. The provided XRD data illustrates the diffraction pattern for compositions at different loadings of itraconazole. More specifically, FIG. 3 illustrates, from the top of the figure to the bottom intensity vs. 2Theta values for, 1) pure itraconazole (ITC pure), 2) physical mixture (PM) of PetOx and itraconazole having 5% w/w of itraconazole, 3) solvent casted (SC) composition of PetOx and itraconazole having 55% w/w of itraconazole, and ASDs of PetOx obtained in accordance with the present invention having 4) 55% w/w itraconazole and 5) 60% w/w itraconazole. Based on the obtained data the 5% w/w ITC-PetOx physical mixture already shows some small peaks related to the pure itraconazole spectrum. For the 55% w/w solvent casted sample clear Bragg peaks are noticeable and hence partial crystallinity. For both 55% w/w itraconazole and 5) 60% w/w itraconazole ASDs amorphous halo is present and no distinct Bragg peaks are ASD seen, hence fully amorphous material were obtained.

The results of the non-sink in vitro dissolution tests are seen in FIG. 4 and indicate a pronounced improvement compared to the dissolution of the crystalline ITC. Samples containing 50 mg of itraconazole were tested in a dissolution bath containing 200 ml of 0.1 M HCl. Dissolution rates are a lot faster and over 50 times more ITC is taken up, as shown in Table 3.

TABLE 3

| | mg ITC dissolved | | | |
|---|---|---|---|---|
| Sample | 5 min | 15 min | 60 min | 300 min |
| Crystalline ITC | 0.37 ± 0.12 | 0.45 ± 0.03 | 0.46 ± 0.04 | 0.50 ± 0.06 |
| 40 wt % ITC ASD | 1.23 ± 0.32 | 3.12 ± 0.94 | 8.96 ± 2.71 | 23.03 ± 3.28 |
| 50 wt % ITC ASD | 1.21 ± 0.02 | 3.24 ± 0.28 | 10.31 ± 1.22 | 25.12 ± 2.13 |
| 60 wt % ITC ASD | 2.18 ± 0.01 | 5.13 ± 0.31 | 12.83 ± 0.51 | 24.91 ± 0.11 |
| 70 wt % ITC ASD | 2.66 ± 0.49 | 5.98 ± 0.88 | 13.58 ± 1.47 | 28.75 ± 4.72 |

More specifically, FIG. 4 illustrates dissolution data of pharmaceutical compositions comprising solution electrospun fibers obtained in accordance with the present invention wherein itraconazole is the active ingredient and PetOx is the polymer. The provided dissolution data illustrates the itraconazole release for compositions at different loadings of itraconazole. More specifically, FIG. 4 illustrates the cumulative amount of itraconazole released at certain time points during the test. Data is shown for pure itraconazole (square) as well as for ASDs of itraconazole-PetOx obtained in accordance with the present invention having 40% w/w itraconazole (downwards triangle), 50% w/w itraconazole (square), 60% w/w itraconazole (circle) and 70% w/w itraconazole (downwards triangle). Based on the obtained data a significantly improved release rate is obtained for the itraconazole-PetOx ASDs obtained in accordance with the present invention. Moreover a significantly increased amount of itraconazole is released compared to pure itraconazole.

### Example 3: Electrospinning of poly(2-ethyl-2-oxazoline) with Celecoxib (CCX)

Amorphous CCX was obtained via solution electrospinning with PetOx as polymeric excipient. Table 4 shows that high drug loadings up to 80 wt % were achieved when an acetone/acetic acid (8:2) solvent system was used (X denotes an inhomogeneous solution,—denotes an inspinnable solution due to a too low solution viscosity). Parameters used for the solvent electrospinning process are a tip to collector distance of 15 cm, a flow rate between 1.5 and 2.5 ml/h and a voltage of 15 kV. Note that the high celecoxib loadings were only reached when a PetOx wt % was used which is lower compared to the minimal required polymer concentration for solution electrospinning pure PetOx.

TABLE 4

| | 20 | 30 | 40 | 50 | 55 | 60 | 65 | 70 | 75 | 80 |
|---|---|---|---|---|---|---|---|---|---|---|
| Celecoxib | wt % | wt % | wt % | wt % | wt % | wt % | wt % | wt % | wt % | wt % |
| 15 wt % | — | — | | | | | | 723 ± | 1269 ± | 1630 ± |
| PetOx | | | | | | | | 129 | 298 | 250 |
| | | | | | | | | nm | nm | nm |
| 20 wt % | | | 741 ± | 1030 ± | 1140 ± | 1320 ± | 1290 ± | 1481 ± | 1778 ± | X |
| PetOx | | | 117 | 150 | 150 | 210 | 539 | 202 | 651 | |
| | | | nm | nm | nm | nm | nm | nm | nm | |

Thermal analysis via MDSC shows no sign of a crystalline endotherm, only a single T_{g} is observed in between −20° C. and 220° C. As shown in FIG. 5 there is a distinct positive deviation of the obtained T_{g} values compared the theoretical T_{g} as calculated by the Fox equation. This positive deviation indicates interaction between PetOx and CCX, positively influencing the stability of the amorphous solid dispersions.

FIG. 5 illustrates MDSC data of pharmaceutical compositions comprising solution electrospun fibers obtained in accordance with the present invention wherein celecoxib is the active ingredient and PetOx is the polymer. The provided MDSC data illustrates the T_{g} values for compositions at different loadings of celecoxib. More specifically, FIG. 5 illustrates the obtained T_{g} values (squares) compared to the theoretically predicted T_{g} values as predicted by the Fox equation for mixing (line). Based on the obtained data values above the prediction are obtained indicating a stability increasing interaction between PetOx and celecoxib. Analyzing the amorphous nature of the solid dispersions via XRD corroborates the amorphicity of the PetOx-CCX material. FIG. 6 compares the crystalline material to the solid dispersions and even for the 65 wt % sample no crystalline Bragg peaks can be observed.

FIG. 6 illustrates XRD data of pharmaceutical compositions comprising solution electrospun fibers obtained in accordance with the present invention wherein celecoxib is the active ingredient and PetOx is the polymer. The provided XRD data illustrates the diffraction pattern for compositions at different loadings of celecoxib. More specifically, FIG. 6 illustrates, from the top of the figure to the bottom intensity vs. 2Theta values for, 1) pure celecoxib (CCX pure), 2) physical mixture (PM) of PetOx and celecoxib having 5% w/w of celecoxib, 3) solvent casted (SC) composition of PetOx and celecoxib having 40% w/w of celecoxib, and ASDs of PetOx obtained in accordance with the present invention having 4) 50% w/w celecoxib and 5) 65% w/w celecoxib. Based on the obtained data, the 5% w/w CCX-PetOx physical mixture already shows some small peaks related to the pure CCX spectrum. For 40% w/w solvent casted sample, an amorphous halo is present, without any signs of Bragg peaks showing up, hence an amorphous material was formed. For ASDs having 50% w/w and 65% w/w in accordance with the present invention, an amorphous halo is present, without any signs of Bragg peaks showing up, hence a fully amorphous material was obtained even at really high drug loadings (65% w/w).

Finally, the non-sink in vitro dissolution testing illustrates the increased dissolution rates compared to crystalline CCX, as summarized in Table 5, over 10 times more CCX gets dissolved when in its amorphous solid state. Samples containing 50 mg of celecoxib were tested in a dissolution bath containing 200 ml of 0.1 M HCl.

TABLE 5

| | mg CCX dissolved | | | |
|---|---|---|---|---|
| Sample | 5 min | 15 min | 60 min | 300 min |
| Crystalline CCX | 0.05 ± 0.05 | 0.10 ± 0.08 | 0.13 ± 0.12 | 0.18 ± 0.12 |
| 40 wt % CCX ASD | 2.03 ± 0.97 | 2.40 ± 0.27 | 2.55 ± 0.07 | 2.35 ± 0.08 |
| 50 wt % CCX ASD | 0.52 ± 0.18 | 1.74 ± 0.27 | 2.09 ± 0.12 | 2.12 ± 0.07 |
| 70 wt % CCX ASD | 0.18 ± 0.06 | 0.37 ± 0.09 | 1.28 ± 0.12 | 2.04 ± 0.01 |
| 80 wt % CCX ASD | 0.23 ± 0.04 | 0.67 ± 0.18 | 1.95 ± 0.29 | 2.13 ± 0.11 |

### Example 4: Electrospinning of poly(2-ethyl-2-oxazoline) with Flubendazole (Flu)

Various nanofibrous amorphous solid dispersions were obtained by the solution electrospinning of PetOx and Flu in a formic acid solvent system. Table 6 shows the ranges of high drug loadings that are obtained (X denotes an inhomogeneous solution,—denotes an inspinnable solution due to a too low solution viscosity). The process conditions were a tip to collector distance of 15 cm, a flow rate ranging between 0.1 and 0.15 ml/h and a voltage of 22.5-30 kV. Note that the high flubendazole loadings were only reached when a PetOx wt % was used which is lower compared to the minimal required polymer concentration for solution electrospinning pure PetOx.

TABLE 6

| Flubendazole | 20 wt % | 30 wt % | 40 wt % | 50 wt % | 55 wt % | 60 wt % |
|---|---|---|---|---|---|---|
| 10 wt % | — | — | — | | | 96 ± 30 |
| PetOx | | | | | | nm |
| 15 wt % | — | — | 107 ± 21 | 177 ± 45 | 231 ± 52 | X |
| PetOx | | | nm | nm | nm | |
| 20 wt % | 137 ± 31 | 127 ± 23 | 207 ± 78 | X | X | X |
| PetOx | nm | nm | nm | | | |
| 25 wt % | 246 ± 57 | 532 ± 183 | X | X | X | X |
| PetOx | nm | nm | | | | |

MDSC analysis reveals that within 0° C. and 120° C. no crystalline endotherm is observed. However a single T_{g} is found indicating a single phase, homogeneous material. Moreover, the T_{g} values found can be fitted according the Gordon-Taylor equation with a fitting parameter, k, of 0.42. XRD analysis on the other hand shows both fully amorphous PetOx-Flu solid dispersion as well as a time stability of 1 year for the amorphous nature of the 25 wt % sample, as can be seen in FIG. 7b. FIG. 7c, shows the diffraction pattern of several physical mixtures in order to observe at what wt % a crystalline fraction of Flu is noticeable. From the results we conclude that starting from 3 wt % crystalline Flu a Bragg peak at 6.5° is showing up. Any sample with a peak at that angle will have at least 3 wt % of crystalline Flu present. Note that for FIGS. 7a and b this is not the case, no crystallinity is detected. This is however the case for solvent casted solid dispersions starting from 25 wt % as indicated in FIG. 7d.

FIG. 7 illustrates XRD data of pharmaceutical compositions comprising solution electrospun fibers obtained in accordance with the present invention wherein flubendazole is the active ingredient and PetOx is the polymer. The provided XRD data illustrates the diffraction pattern for compositions at different loadings of flubendazole. More specifically, FIG. 7a illustrates, from the top of the figure to the bottom intensity vs. 2Theta values for, ASDs of PetOx obtained in accordance with the present invention having 1) 55% w/w of flubendazole, 2) 50% w/w of flubendazole, 3) 30% w/w of flubendazole, 4) 25% w/w of flubendazole and 5) 15% w/w of flubendazole, 6) pure PetOx nanofibers and 7) pure flubendazole (Flu). FIG. 7b illustrates, from the top of the figure to the bottom intensity vs. 2Theta values for, ASDs of PetOx obtained in accordance with the present invention having 1) one year old 25% w/w of flubendazole ASD, 2) one month old 25% w/w of flubendazole ASD, 3) pure PetOx nanofibers and 4) pure flubendazole (Flu). FIG. 7c illustrates, from the top of the figure to the bottom intensity vs. 2Theta values for, physical mixture (PM) of PetOx and flubendazole having 1) 15% w/w of flubendazole, 2) 10% w/w of flubendazole, 3) 5% w/w of flubendazole, 4) 3% w/w of flubendazole, 5) 1% w/w of flubendazole and 6) pure flubendazole (Flu). FIG. 7d illustrates, from the top of the figure to the bottom intensity vs. 2Theta values for, solvent casted (SC) composition of PetOx and flubendazole having 1) 25% w/w of flubendazole, 2) 20% w/w of flubendazole, 3) 15% w/w of flubendazole, 4) pure solvent casted PetOx, 5) pure PetOx nanofibers and 6)) pure flubendazole. Based on the obtained data, the 3% w/w Flu-PetOx physical mixture already shows some small peaks related to the pure flubendazole spectrum (FIG. 7d). For 20% w/w solvent casted sample and higher, a clear Bragg peak is noticeable and hence partial crystallinity is present (FIG. 7d). For all ASDs in accordance with the present invention, an amorphous halo is present, without any signs of Bragg peaks showing up, hence a fully amorphous material was obtained even at really high drug loadings (55% w/w) (FIGS. 7a and b). Moreover, the amorphous stability is confirmed to be stable for at least one year for the 25% w/w, as the diffraction pattern is unchanged and remained without any signs of Bragg peaks (FIG. 7b).

This time stability can also be observed by the sink in vitro dissolution tests. FIG. 8a shows the results of all samples compared to the crystalline Flu, while FIG. 8b shows the 25 wt % PetOx-Flu sample 1 month vs. 1 year old behavior. It is clear that no significant difference is observed in the Flu release after 1 year of storage under 25° C. and 25% RH. Table 7 and 8 give an overview of the dissolution rate after 10 minutes for the samples shown in FIG. 8a and FIG. 8b respectively. Samples containing 50 mg of flubendazole were tested in a dissolution bath containing 900 ml of 0.1 M HCl.

TABLE 7

| | Dissolution rate (10 min) |
|---|---|
| Sample | (μg mL⁻¹ min⁻¹) |
| Flu | 0.07 ± 0.03 |
| PetOx-15 wt % Flu | 0.26 ± 0.03 |
| PetOx-20 wt % Flu | 0.29 ± 0.07 |
| PetOx-25 wt % Flu | 0.31 ± 0.03 |
| PetOx-30 wt % Flu | 0.27 ± 0.06 |
| PetOx-35 wt % Flu | 0.32 ± 0.04 |
| PetOx-40 wt % Flu | 0.37 ± 0.04 |
| PetOx-45 wt % Flu | 0.27 ± 0.07 |
| PetOx-50 wt % Flu | 0.30 ± 0.02 |
| PetOx-55 wt % Flu | 0.24 ± 0.01 |

TABLE 8

| | Dissolution rate (10 min) |
|---|---|
| Sample | (μg mL⁻¹ min⁻¹) |
| Flu | 0.07 ± 0.03 |
| PetOx-25 wt % Flu (1 week) | 0.31 ± 0.03 |
| PetOx-25 wt % Flu (1 year) | 0.30 ± 0.08 |

FIG. 8 illustrates dissolution data of pharmaceutical compositions comprising solution electrospun fibers obtained in accordance with the present invention wherein flubendazole is the active ingredient and PetOx is the polymer. The provided dissolution data illustrates the itraconazole release for compositions at different loadings of flubendazole. More specifically, FIG. 8a illustrates the cumulative amount of flubendazole released at certain time points during the test. Data is shown for pure, crystalline, flubendazole as well as for ASDs of flubendazole-PetOx obtained in accordance with the present invention having 20% w/w flubendazole (upwards triangle), 30% w/w flubendazole (square), 40% w/w flubendazole (void upwards triangle), 50% w/w flubendazole (void circle) and 55% w/w flubendazole (leftwards triangle). FIG. 8b illustrates the cumulative amount of flubendazole released at certain time points during the test. Data is shown for pure, crystalline, flubendazole (downwards triangle) as well as for ASDs of flubendazole-PetOx obtained in accordance with the present invention having 25% w/w flubendazole being one week old and 25% w/w flubendazole being one year old. Based on the obtained data a significantly improved release rate is obtained for the flubendazole-PetOx ASDs obtained in accordance with the present invention. Moreover a significantly increased amount of flubendazole is released compared to pure flubendazole as a full flubendazole release is obtained within 300 min for the solution electrospun flubendazole-PetOx ASDs (FIG. 8a). The ASD stability is further confirmed to be stable for at least one year for the 25% w/w, as the release profile remained unchanged and a full flubendazole release was obtained for both samples being one week and one year old.

### Example 5: Electrospinning of polyvinylpyrrolidon K30 with Flubendazole

Various samples were solution electrospun in a formic acid/acetic acid (9:1) solvent system. Conditions used for the electrospinning process were a 25 wt % of PVP K30 present in the solution, a tip to collector distance of 15 cm, a flow rate of 0.1-0.5 ml/h and a voltage ranging from 28-30 kV. FIG. 9 illustrates that a certain amount of Flu is required in order to obtain a uniform nanofibrous membrane. Without Flu present in the solution a beaded membrane is obtained (FIG. 9a), whereas a uniform membrane is formed when 20 wt % Flu is present (FIG. 9b).

FIG. 9 illustrates SEM images comprising solution electrospun fibers obtained in accordance with the present invention wherein flubendazole is the active ingredient and PVP K30 is the polymer. More specifically FIG. 9a illustrates the presence of beads in the membrane due to a viscosity/polymer concentration, 25% w/w PVP K30, below the minimal amount required for a stable and uniform solution electrospinning process. FIG. 9b illustrates a uniform solution electrospun nanofibrous membrane, electrospun from a 25% w/w PVP K30-20% w/w flubendazole solution. Based on the obtained data the effect of a certain flubendazole concentration on the electrospinability is observed.

### Example 6: Electrospinning of polyvinylpyrrolidon K90 with Flubendazole

Various samples were solution electrospun in a formic acid solvent system. Conditions used for the electrospinning process of PVP K90 and flubendazole were a tip to collector distance of 15 cm, a flow rate of 0.1-0.5 ml/h and a voltage ranging from 20-28 kV. Table 9 shows the ranges of drug loadings that are obtained (X denotes an inhomogeneous solution,—denotes an inspinnable solution due to a too low solution viscosity). Note that the high flubendazole loadings were only reached when a PVP K90 wt % was used which is lower compared to the minimal required polymer concentration for solution electrospinning pure PVP K90.

FIG. 10 illustrates SEM images comprising solution electrospun fibers obtained in accordance with the present invention wherein flubendazole is the active ingredient and PVP K90 is the polymer. More specifically FIG. 10a illustrates the presence of beads in the membrane due to a viscosity/polymer concentration, 7.5% w/w PVP K90, below the minimal amount required for a stable and uniform solution electrospinning process. FIG. 10b illustrates a uniform solution electrospun nanofibrous membrane, electrospun from a 7.5% w/w PVP K90-50% w/w flubendazole solution. Based on the obtained data the effect of a certain flubendazole (also referred as “Flu”) concentration on the electrospinability is observed.

TABLE 9

| | 0 | 15 | 20 | 25 | 30 | 35 | 40 | 45 | 50 | 60 | 70 |
|---|---|---|---|---|---|---|---|---|---|---|---|
| Flu | wt % | wt % | wt % | wt % | wt % | wt % | wt % | wt % | wt % | wt % | wt % |
| 7.5 | — | 274 ± | 240 ± | 166 ± | 203 ± | 196 ± | 234 ± | 300 ± | 285 ± | 291 ± | X |
| wt % | | 61 | 58 | 25 | 60 | 61 | 64 | 120 | 62 | 143 | |
| PVP | | nm | nm | nm | nm | nm | nm | nm | nm | nm | |
| K90 | | | | | | | | | | | |
| 10 | 374 ± | 434 ± | 564 ± | 359 ± | 400 ± | 478 ± | 420 ± | 631 ± | 471 ± | X | X |
| wt % | 76 | 152 | 198 | 95 | 163 | 167 | 99 | 242 | 141 | | |
| PVP | nm | nm | nm | nm | nm | nm | nm | nm | nm | | |
| K90 | | | | | | | | | | | |

### Example 7: Electrospinning of poly(2-ethyl-2-oxazoline) with Ultra-High Flubendazole (Flu) Loading

A 10% w/w PEtOx-65% w/w flubendazole nanofibrous amorphous solid dispersion was obtained by the solution electrospinning of PEtOx and Flu in a formic acid solvent system. The process conditions were a tip to collector distance of 15 cm, a flow rate of 0.1 ml/h and a voltage of 27 kV. Note that this high flubendazole loading was only reached when a PEtOx wt % was used which is lower compared to the minimal required polymer concentration for solution electrospinning pure PEtOx, as shown in Example 4.

FIG. 11 illustrates XRD data of pharmaceutical compositions comprising solution electrospun fibers obtained in accordance with the present invention wherein flubendazole is the active ingredient and PEtOx is the polymer. The provided XRD data illustrates the diffraction pattern for compositions at different loadings of flubendazole. More specifically, FIG. 11 illustrates, from the top of the figure to the bottom intensity vs. 2Theta values for, ASDs of PEtOx obtained in accordance with the present invention having 1) 65% w/w of flubendazole, 2) 55% w/w of flubendazole, 3) 50% w/w of flubendazole, 4) 30% w/w of flubendazole, 5) 25% w/w of flubendazole and 6) 15% w/w of flubendazole, 7) pure PEtOx nanofibers and 8) pure flubendazole (Flu). For all ASDs in accordance with the present invention, an amorphous halo is present, without any signs of Bragg peaks showing up, hence a fully amorphous material was obtained even at really high drug loadings (65% w/w) (FIG. 11).

### Example 8: Electrospinning of a Time-Stable poly(2-ethyl-2-oxazoline) with High Flubendazole (Flu) Loading

A 15% w/w PEtOx-50% w/w flubendazole nanofibrous amorphous solid dispersion was obtained by the solution electrospinning of PEtOx and Flu in a formic acid solvent system. The process conditions were a tip to collector distance of 15 cm, a flow rate of 0.12 ml/h and a voltage of 27.5 kV. Note that this high flubendazole loading was only reached when a PEtOx wt % was used which is lower compared to the minimal required polymer concentration for solution electrospinning pure PEtOx, as shown in Example 4.

FIG. 12 illustrates XRD data of this pharmaceutical composition comprising solution electrospun fibers obtained in accordance with the present invention wherein flubendazole is the active ingredient and PEtOx is the polymer. The provided XRD data illustrates the diffraction pattern for the 15% w/w PEtOx-50% w/w flubendazole nanofibrous amorphous solid dispersion at different storage times. More specifically, FIG. 12 illustrates, from the top of the figure to the bottom intensity vs. 2Theta values for, ASDs of PEtOx obtained in accordance with the present invention having 1) 2.5 years old 50% w/w of flubendazole ASD, 2) one month old 50% w/w of flubendazole ASD, 3) pure PEtOx nanofibers and 4) pure flubendazole (Flu). For all ASDs in accordance with the present invention, an amorphous halo is present, without any signs of Bragg peaks showing up, hence a fully amorphous material was obtained (FIG. 12). Moreover, the amorphous stability is confirmed to be stable for at least 2.5 years for the 50% w/w, as the diffraction pattern remained without any signs of Bragg peaks (FIG. 12).

### Example 9: In Vivo Assessment of Electrospun poly(2-ethyl-2-oxazoline) with High Flubendazole (Flu) Loading

A 15% w/w PEtOx-50% w/w flubendazole nanofibrous amorphous solid dispersion was obtained by the solution electrospinning of PEtOx and Flu in a formic acid solvent system. The process conditions were a tip to collector distance of 15 cm, a flow rate of 0.12 ml/h and a voltage of 27.5 kV. Note that this high flubendazole loading was only reached when a PEtOx wt % was used which is lower compared to the minimal required polymer concentration for solution electrospinning pure PEtOx, as shown in Example 4. For analysis of the in vivo bioavailability, a study with Beagle dogs was set up and approved by the ethics committee of Ghent University. Plasma concentrations of Flu and its two metabolites, namely NH2-Flu (hydrolized Flu; H-Flu) or OH-Flu (reduced Flu; R-Flu) were examined after either PEtOx-Flu ASD or pure Flu intake. Table 10 gives an overview of Cₘₐₓ, tₘₐₓ and area under the curve (AUC) ratio values (ratio of the AUC of ASD over the AUC of pure Flu) after oral administration of crystalline Flu and a 50 wt % PEtOx-Flu ASD to Beagle dogs, n=5. Concentrations of Flu and its two metabolites, H-Flu and R-Flu, were monitored.

FIG. 13 illustrates the average plasma concentration of a 15% w/w PEtOx-50% w/w flubendazole pharmaceutical composition comprising solution electrospun fibers obtained in accordance with the present invention wherein flubendazole is the active ingredient and PEtOx is the polymer. The provided in vivo data illustrates the average plasma concentration for different metabolites of flubendazole. More specifically, FIG. 13a illustrates, Flu concentration vs. time for, ASDs of PEtOx obtained in accordance with the present invention having 1) 50% w/w of flubendazole, 2) pure flubendazole (Flu). FIG. 13b illustrates, H-Flu concentration vs. time for, ASDs of PEtOx obtained in accordance with the present invention having 1) 50% w/w of flubendazole, 2) pure flubendazole (Flu). FIG. 13c illustrates, R-Flu concentration vs. time for, ASDs of PEtOx obtained in accordance with the present invention having 1) 50% w/w of flubendazole, 2) pure flubendazole (Flu). It is evident from FIG. 13 that the nanofibrous ASDs outperform the pure Flu.

FIG. 14 illustrates the AUC of a 15% w/w PEtOx-50% w/w flubendazole pharmaceutical composition comprising solution electrospun fibers obtained in accordance with the present invention wherein flubendazole is the active ingredient and PEtOx is the polymer. The provided in vivo data illustrates the AUC for different metabolites of flubendazole. More specifically. FIG. 14a illustrates, Flu AUC for, ASDs of PEtOx obtained in accordance with the present invention having 1) 50% w/w of flubendazole, 2) pure flubendazole (Flu). FIG. 14b illustrates, H-Flu AUC for, ASDs of PEtOx obtained in accordance with the present invention having 1) 50% w/w of flubendazole, 2) pure flubendazole (Flu). FIG. 14c illustrates, R-Flu AUC for, ASDs of PEtOx obtained in accordance with the present invention having 1) 50% w/w of flubendazole, 2) pure flubendazole (Flu). It is evident from FIG. 14 and Table 10 that on average the 50 wt % nanofibrous ASD led to higher bioavailability of Flu compared to administration of the pure Flu.

The AUC ratio in Table 10 indicates that the ASD resulted in up to four times higher bioavailability compared to pure Flu. Additionally, when comparing Cₘₐₓ values in Table 10, it is clear that the ASDs outperform crystalline Flu. A paired t-test on the AUC values indicates a significant difference between the bioavailability of ASD-Flu and pure Flu, p<0.1 for Flu and p<0.05 for H-Flu and R-Flu.

TABLE 10

| | | Cₘₐₓ (ng/ml) | tₘₐₓ (h) | AUC ratio |
|---|---|---|---|---|
| Flu | 50 wt % Flu ASD | 4.7 ± 3.8 | 1.4 ± 0.5 | 2.3 ± 0.8 |
| | Pure Flu | 1.2 ± 0.4 | 1 ± 0.6 | |
| H-Flu | 50 wt % Flu ASD | 40.3 ± 23.5 | 1.8 ± 0.4 | 4.1 ± 2.5 |
| | Pure Flu | 7.5 ± 12.6 | 7.4 ± 6.5 | |
| R-Flu | 50 wt % Flu ASD | 15.7 ± 9.4 | 1.6 ± 0.5 | 4.0 ± 1.4 |
| | Pure Flu | 1.6 ± 0.6 | 5.4 ± 6.0 | |

### REFERENCES

1. Vrbata, Petr, et al. “Electrospun drug loaded membranes for sublingual administration of sumatriptan and naproxen.” International journal of pharmaceutics 457.1 (2013): 168-176.

1. A process of manufacturing a solution electrospun fiber, comprising the steps of:
a) providing a solution comprising an ingredient, a polymer and a solvent;
b) electrospinning the solution provided at step a) thereby obtaining a solution electrospun fiber comprising an amorphous form of the ingredient;
wherein step a) comprises providing the polymer at a concentration in said solution which is lower than the concentration at which the polymer is solution electrospinnable into fibers in the absence of the ingredient.

2. The process according to claim 1, wherein at step a) the ingredient is provided in an amount of at least 50%, preferably above 50% w/w of ingredient to polymer.

3. The process according to any one of the previous claims, wherein at step a) the polymer is a poly(2-alkyl-2-oxazoline) (PAOx), preferably poly(2-ethyl-2-oxazoline) (PEtOx).

4. The process according to claim 3, wherein the polymer is poly(2-ethyl-2-oxazoline) (PEtOx).

5. The process according to any one of the previous claims, wherein at step a) the ingredient and the polymer are selected from those providing a mixture consisting of said ingredient and polymer yielding a solution electrospun fiber having a glass transition temperature T_{g} of at least about 25° C.

6. The process according to any one of the previous claims, wherein at step a) the ingredient is provided in an amount of at least 55% w/w, at least 60% w/w, at least 65% w/w, at least 70% w/w of ingredient to polymer.

7. The process according to any one of the previous claims, wherein at step a) the polymer is a water-soluble polymer.

8. The process according to any one of the previous claims, wherein at step a) the solvent comprises an acid.

9. A solution electrospun fiber obtainable by means of the method defined in claims 1 to 8, comprising:
an ingredient in an amorphous form;
a polymer;
wherein the ingredient is in an amount of at least 50%, preferably above 50% w/w of ingredient to polymer, preferably in an amount of at least 55% w/w, at least 60% w/w, at least 65% w/w, at least 70% w/w.

10. A solution electrospun fiber comprising:
an ingredient in an amorphous form;
a polymer;
wherein the solution electrospun fiber is electrospun from a polymer solution comprising a polymer at a concentration in said solution which is lower than the concentration at which the polymer is solution electrospinnable in the absence of the ingredient.

11. The fiber according to claim 10, wherein the ingredient is in an amount of at least 50%, above 50% w/w of ingredient to polymer, preferably in an amount of at least 55% w/w, at least 60% w/w, at least 65% w/w, at least 70% w/w.

12. The fiber according to any one of claims 9 to 11, wherein the polymer is a poly(2-alkyl-2-oxazoline) (PAOx), preferably poly(2-ethyl-2-oxazoline) (PEtOx).

13. The fiber according to any one of claims 9 to 12, wherein the polymer is an amorphous polymer.

14. The fiber according to any one of claims 9 to 13, wherein the ingredient and the polymer are selected from those providing a mixture consisting of said ingredient and polymer yielding a solution electrospun fiber having a glass transition temperature T_{g} of at least about 25° C.

15. A composition comprising a solution electrospun fiber as defined in any one of claims 9 to 14.
